(19) United States
(12) Patent Application Publication
(10) Pub. No.: US 2021/0015755 A1
(43) Pub. Date: Jan. 21, 2021

(54) SECNIDAZOLE SOFT GELATIN CAPSULE AND METHODS AND USES THEREOF

(71) Applicant: Lupin Inc., Baltimore, MD (US)

(72) Inventors: Richard Holl, Somerset, NJ (US); James Garegnani, Somerset, NJ (US); Gregory Kaufman, Somerset, NJ (US)

(73) Assignee: Lupin Inc., Baltimore, MD (US)

(21) Appl. No.: 16/514,586

(22) Filed: Jul. 17, 2019

(51) Int. Cl. A61K 9/48; A61K 31/4164; A61K 9/00

(52) U.S. Cl. CPC A61K 9/4825; A61K 31/4164; A61K 9/0034; A61K 9/4875; A61K 9/4858

(57) ABSTRACT

A soft gelatin capsule composition for vaginal administration of secnidazole includes a therapeutically effective amount of secnidazole dispersed in a pharmaceutically acceptable carrier. The therapeutically effective amount of secnidazole is in a range of from about 1 mg to about 500 mg, or in a range of from about 1,000 mg to about 1,500 mg. The composition is used for treating diseases, including bacterial vaginosis or trichomoniasis.

### FIELD OF THE INVENTION

The present invention relates to novel soft gelatin capsule compositions and formulations for intravaginal administration comprising secnidazole compounds and uses of these pharmaceutical compositions and formulations in the treatment of diseases such as bacterial vaginosis and trichomoniasis.

All references and products cited within this application and their disclosures are incorporated by reference herein in their entirety.

### BACKGROUND

Urogenital infections affect more than one billion women per year, all over the world, representing one of the main reasons for women to seek medical consultation. Bacterial vaginosis (BV) is one of the main causes of vaginal problems, is the cause of malodorous discharge in women of reproductive age, and affects an estimated 10-15% of reproductive aged women. It is also associated with several public health problems, including, preterm birth and acquisition and/or transmission of sexually transmitted diseases (Eschenbach D. A., Clin. Infect. Dis. (1993) 16:S282-287; Hillier S., AIDS Res. Hum. Retroviruses (1998) 14:17-21).

BV is a complex disease and occurs due to change in normal vaginal flora. The precise cause of BV, however, it is not yet clear and it is hypothesized that BV may be caused by an increase in the number of Gardnerella vaginalis and other anaerobes in vagina, along with a decrease in lactobacilli (Hill G. B., Am. J. Obstet. Gynecol. (1993) 169:450-4; Ferris et al., J. Clin. Microbiol. (2004) 42:5892-4). Vaginal biopsy sample studies have revealed that BV consists of a dense biofilm in which G. vaginalis is predominant (Swidsinski et al., Obstet. Gynecol. (2005) 106:1013-1023).

Delivering medications, particularly high doses, intravaginally also poses several challenges. The rate and extent of drug absorption after intravaginal administration depends on several factors such as formulation, vaginal physiology, age of the patient and menstrual cycle of the patient. Further, considerable variability in the rate and extent of absorption of vaginally administered drugs is observed by changes in thickness of vaginal epithelium. Issues such as cultural sensitivity, personal hygiene, gender specificity, local irritation and influence of sexual intercourse, also need to be addressed during the design of a vaginal formulation.

Trichomoniasis is a very common sexually transmitted disease (STD). It is caused by infection with a protozoan parasite called Trichomonas vaginalis. Although symptoms of the disease vary, most people who have the parasite cannot tell they are infected. About 70% of infected people do not have any signs or symptoms. When trichomoniasis does cause symptoms, they can range from mild irritation to severe inflammation. Some people with symptoms get them within 5 to 28 days after being infected. Others do not develop symptoms until much later. Symptoms can come and go. This disease is as an important source of reproductive morbidity, a facilitator of HIV transmission and acquisition, and thus it is an important public health problem. Despite its importance in human reproductive health and HIV transmission, it is not a reportable disease and surveillance is not generally done.

The drug product SECNOL, which has already been approved in France, contains secnidazole for the treatment of BV and trichomoniasis. Nitroimidazoles have been extensively evaluated in the treatment of trichomoniasis, giardiasis, liver and intestinal amoebiasis. The most widely used are metronidazole, tinidazole, ornidazole, and secnidazole (“Nitroimidazole in the treatment of Trichomoniasis, giardiasis and amebiasis,” International Journal of Clinical Pharmacology Therapy and Toxicology (1984) 22(2): 63-72).

The emerging resistance of Trichomonas vaginalis to metronidazole and the reliance on a single class of antimicrobial drugs warrants the evaluation of existing compounds and development of novel systemic treatment options. Secnidazole, administered as a single dose, with a longer half-life and relatively fewer adverse effects, may be useful as an adjunct or alternative to metronidazole, especially for patients with metronidazole-resistant Trichomonas vaginalis infections. (Arindam P Ghosh, Cheri Aycock and Jane R Schwebke, “Susceptibility of clinical isolates of Trichomonas vaginalis to metronidazole and secnidazole—an in vitro study,” Antimicrob Agents Chemother, 2018 Mar. 27; 62(4). pii: e02329-17. doi: 10.1128/AAC.02329-17. Print 2018 April).

Secnidazole, a second generation 5-nitroimidazole, is an antiprotozoal and anti-microbial drug (Gillis and Wiseman, “Secnidazole: A Review of its Antimicrobial Activity, Pharmacokinetic Properties, and Therapeutic Use in the Management of Protozoal Infections and Bacterial Vaginosis,” Drugs, 51(4), 621-638 (1996)). Secnidazole is particularly effective in treatment of amoebiasis, giardiasis, trichomoniasis, and bacterial vaginosis. It is rapidly and completely absorbed after oral administration and has a terminal elimination half-life of about 17 to about 29 hours.

Secnidazole enters in to bacterial cell as a prodrug without antimicrobial activity and is converted into its active form in vivo via reduction of its nitro group to radical anions by bacterial enzymes. The radical anions are thought to interfere with bacterial DNA synthesis resulting in the antimicrobial activity. Because of its antimicrobial activity and long terminal elimination half-life, secnidazole can be administered as a single dose for the treatment of bacterial vaginosis (Videau et al., “Secnidazole: A 5-nitroimidazole Derivative With a Long Half-life,” Brit. J. Venereal Diseases, 54, 77-80 (1978); Gillis and Wiseman, “Secnidazole: A Review of its Antimicrobial Activity, Pharmacokinetic Properties, and Therapeutic Use in the Management of Protozoal Infections and Bacterial Vaginosis,” Drugs, 51(4), 621-638 (1996); Bohbot et al., “Treatment of Bacterial Vaginosis: A Multicenter, Double-Blind, Double-Dummy, Randomised Phase III Study Comparing Secnidazole and Metronidazole,” Infect. Dis. Obstet. Gynecol. vol. 2010, Article ID 705692, 6 pages, (2010)). Secnidazole is mainly metabolized via hydroxylation and glucuroconjugation in the liver and 50% of secnidazole is excreted intact in the urine (Frydman et al., “A Review of the Pharmacokinetics of Secnidazole in Man,” 16^{th} International Congress of Chemotherapy, 2, 445.1-445.3 (1989)).

In 2017, SOLOSEC® (secnidazole, 2 g oral granules) was approved for the treatment of bacterial vaginosis in adult women by the Food and Drug Administration (FDA). SOLOSEC® has enhanced pharmacokinetic properties that enable its delivery and efficacy in a single dose. There are, however, certain adverse reactions reported for SOLOSEC®, such as, vulvovaginal candidiasis, headache, nausea, dysgeusia, vomiting, diarrhea, abdominal pain, and vulvovaginal pruritus (see Prescribing Information for SOLOSEC®).

Clinical research involving the use of 5-nitroimidazole antimicrobial compounds other than secnidazole indicate that lower doses from these 5-nitroimidazole antimicrobial compounds administered vaginally may be efficacious compared to these compounds administered orally and produces less gastrointestinal side-effects. In addition, 5-nitroimidazole antimicrobial compounds administered vaginally may exhibit a more pronounced local effect than these compounds administered orally, thus increasing their therapeutic effectiveness.

Also, vaginal administration of metronidazole gel is not recommended for treating trichomoniasis because metronidazole gel does not reach therapeutic levels in the urethra and perivaginal glands. Because it is less efficacious than oral metronidazole, metronidazole vaginal gel is not recommended for treating trichomoniasis according to CDC 2015 treatment guide.

U.S. Patent Application Publication No. 20190008784 A1 discloses a novel secnidazole soft gelatin capsule formulation and uses of these pharmaceutical compositions in the treatment of bacterial vaginosis. The patent application discloses embodiments specifying a therapeutically effective amount of secnidazole in the soft gelatin capsule of between 500 mg to 1,000 mg, or about 750 mg.

Vaginal administration of 5-nitroimidazole antimicrobial compounds in the form of gels or creams can result in inconsistent delivery and patient inconvenience. Vaginal administration of 5-nitroimidazole antimicrobial compounds in the form of suppositories as defined as solid dosage forms comprising hard fat or waxes that melt at body temperature after administration have been developed. However, patients frequently complain that these formulations are messy and inconvenient to use.

### SUMMARY

In consideration of the above problems, in accordance with one aspect disclosed herein, soft gelatin capsule compositions for vaginal administration of secnidazole comprising a therapeutically effective amount of secnidazole dispersed in a carrier are disclosed. The carrier can be an excipient comprising a fatty acid derivative, which can be a derivative of a poly-unsaturated fatty acid, a mono-unsaturated fatty acid, or a saturated fatty acid. The therapeutically effective amount of secnidazole is dispersed in such a carrier, with or without in the inclusion of viscosity modifying agents.

In accordance with another aspect disclosed herein, a method for treating bacterial vaginosis in a subject in need thereof. The method includes selecting the subject in need of treatment for bacterial vaginosis and administering, to the subject, a pharmaceutical composition including a therapeutically effective amount of secnidazole, pharmaceutically acceptable secnidazole salt, or pharmaceutically acceptable secnidazole hydrate or solvate. The amount of secnidazole or a pharmaceutically acceptable salt thereof in the pharmaceutical composition is in a range of from about 1 mg to about 500 mg or other ranges thereinbetween (e.g., from about 1 mg to about 499 mg; from about 1 mg to about 450 mg; from about 1 mg to about 400 mg; from about 1 mg to about 300 mg; from about 1 mg to about 200 mg; from about 1 mg to about 100 mg) and the pharmaceutical composition is in the form of a vaginal soft gelatin capsule.

In accordance with another aspect disclosed herein, a method for treating bacterial vaginosis in a subject in need thereof. The method includes selecting the subject in need of treatment for bacterial vaginosis and administering to the subject a pharmaceutical composition including a therapeutically effective amount of secnidazole, pharmaceutically acceptable secnidazole salt, or pharmaceutically acceptable secnidazole hydrate or solvate. The amount of secnidazole or a pharmaceutically acceptable salt thereof in the pharmaceutical composition is in a range of from about 1,000 mg to about 1,500 mg or other ranges thereinbetween (e.g., from about 1,001 mg to about 1,500 mg; from about 1,100 mg to about 1,500 mg; from about 1,200 mg to about 1,500 mg; from about 1,300 mg to about 1,500 mg; from about 1,400 mg to about 1,500 mg) and the pharmaceutical composition is in the form of a vaginal soft gelatin capsule.

In accordance with another aspect disclosed herein, a method for treating trichomoniasis in a subject in need thereof. The method includes selecting the subject in need of treatment for trichomoniasis and administering to the subject a pharmaceutical composition including a therapeutically effective amount of secnidazole or pharmaceutically acceptable salt thereof. The amount of secnidazole or a pharmaceutically acceptable salt thereof in the pharmaceutical composition is in a range of from about 1 mg to about 500 mg or other ranges thereinbetween (e.g., from about 1 mg to about 499 mg; from about 1 mg to about 450 mg; from about 1 mg to about 400 mg; from about 1 mg to about 300 mg; from about 1 mg to about 200 mg; from about 1 mg to about 100 mg) and the pharmaceutical composition is in the form of a vaginal soft gelatin capsule.

In accordance with another aspect disclosed herein, a method for further treating trichomoniasis in a subject in need thereof. The method includes selecting the subject in need of treatment for trichomoniasis and administering to the subject a pharmaceutical composition including a therapeutically effective amount of secnidazole or pharmaceutically acceptable salt thereof. The amount of secnidazole or a pharmaceutically acceptable salt thereof in the pharmaceutical composition is in a range of from about 1,000 mg to about 1,500 mg or other ranges thereinbetween (e.g., from about 1,001 mg to about 1,500 mg; from about 1,100 mg to about 1,500 mg; from about 1,200 mg to about 1,500 mg; from about 1,300 mg to about 1,500 mg; from about 1,400 mg to about 1,500 mg) and the pharmaceutical composition is in the form of a vaginal soft gelatin capsule.

In accordance with another aspect disclosed herein, a method of manufacturing the pharmaceutical composition by a process known as encapsulation using the rotary die encapsulation process. Manufacturing properties, such as the viscosity of the secnidazole fill material, when the fill material is in a fluid state during processing, will also determine the minimum amount of fill composition that is needed to disperse, mold, and package a soft gelatin capsule having a given amount of secnidazole.

### DETAILED DESCRIPTION

For purposes of the description hereinafter, it is to be understood that the embodiments described below may assume alternative variations and embodiments. It is also to be understood that the specific articles, compositions, and/or processes described herein are exemplary and should not be considered as limiting. The present invention is not limited to particular processes, compounds, or methodologies described, as these may vary. The terminology used in the description is for the purpose of describing particular versions or embodiments only, and is not intended to limit the scope of the present invention. Unless defined otherwise, all technical and scientific terms used herein have the same meanings as commonly understood by one of ordinary skill in the art. Any methods and materials similar or equivalent to those described herein can be used in the practice or testing of embodiments of the invention, the preferred methods, devices, and materials described herein.

In this disclosure the singular forms “a,” “an,” and “the” include the plural reference, and reference to a particular numerical value includes at least that particular value, unless the context clearly indicates otherwise. When values are expressed as approximations, by use of the antecedent “about,” it will be understood that the particular value forms another embodiment. As used herein, “about X” (where X is a numerical value) preferably refers to ±10% of the recited value, inclusive. For example, the phrase “about 8” preferably refers to a value of 7.2 to 8.8, inclusive; as another example, the phrase “about 8%” preferably (but not always) refers to a value of 7.2% to 8.8%, inclusive. Where present, all ranges are inclusive and combinable. For example, when a range of “1 to 5” is recited, the recited range should be construed as including ranges “1 to 4”, “1 to 3”, “1-2”, “1-2 & 4-5”, “1-3 & 5”, “2-5”, and the like. In addition, when a list of alternatives is positively provided, such listing can be interpreted to mean that any of the alternatives can be excluded, e.g., by a negative limitation in the claims. For example, when a range of “1 to 5” is recited, the recited range can be construed as including situations whereby any of 1, 2, 3, 4, or 5 are negatively excluded; thus, a recitation of “1 to 5” can be construed as “1 and 3-5, but not 2”, or simply “wherein 2 is not included.” It is intended that any component, element, attribute, or step that is positively recited herein can be explicitly excluded in the claims, whether such components, elements, attributes, or steps are listed as alternatives or whether they are recited in isolation.

Open terms such as “include,” “including,” “contain,” “containing” and the like mean “comprising.” These open-ended transitional phrases are used to introduce an open ended list of elements, method steps or the like that does not exclude additional, unrecited elements or method steps. It is understood that wherever embodiments are described with the language “comprising,” otherwise analogous embodiments described in terms of “consisting of” and/or “consisting essentially of” are also provided.

The transitional phrase “consisting of” and variations thereof excludes any element, step, or ingredient not recited, except for impurities ordinarily associated therewith.

The transitional phrase “consists essentially of,” or variations such as “consist essentially of” or “consisting essentially of” excludes any element, step, or ingredient not recited except for those that do not materially change the basic or novel properties of the specified method, structure or composition.

The terms “substantial,” “substantially,” and variations thereof as used herein are intended to note that a described feature is equal or approximately equal to a value or description. Moreover, “substantially similar” is intended to denote that two values are equal or approximately equal. In some embodiments, “substantially similar” can denote values within about 10% of each other, such as within about 5% of each other, or within about 2% of each other.

Based on U.S. Patent Application Publication No. 2019/0008784, nitroimidazole drugs such as secnidazole are effective in the treatment of several conditions including bacterial vaginosis.

In some instances, administration of a nitroimidazole directly to the vagina is preferable due to toxicity related with oral administration in certain patients. Studies have shown that higher doses of the nitroimidazole, metronidazole, results in a superior therapeutic effect in the treatment of bacterial vaginosis. In one example, Sanchez et al. (American Journal of Obstetrics and Gynecology (2004) 191:1898-906) demonstrated that 500 mg ovule formulation of metronidazole was significantly more effective than a 37.5 mg metronidazole gel in the treatment of bacterial vaginosis. In another example, Aguin et al. (Journal of Lower Genital Tract Disease, (2014), 18(2):156-161) demonstrated that intravaginal doses of a 750 mg ovule formulation of metronidazole resulted in a higher cure rates than intravaginal doses of a 500 mg ovule composition of metronidazole.

However, these elevated doses of drug are hard to incorporate into a formulation for vaginal administration due to the effect of the large amounts of solid drug on flowability of the excipients used in the manufacture of compositions suitable for vaginal administration. Ovule formulations have been formulated using mineral oils which are capable of carrying high drug loads but the interaction of mineral oil with latex poses problems for patients using contraceptives such as condoms to prevent pregnancy and avoid transmission of sexual transmitted diseases. Vaginal suppositories have been used, but patients frequently complain that these formulations are messy and inconvenient to use.

U.S. Patent Application Publication No. 20190008784 discloses soft gelatin capsule composition for vaginal administration of secnidazole comprising a soft gelatin capsule and a therapeutically effective amount of secnidazole dispersed in an excipient, wherein the therapeutically effective amount of secnidazole is from 500 milligrams to 1,000 milligrams.

Secnidazole is a second-generation 5-nitroimidazole antimicrobial that is structurally related to 5-nitroimidazoles, including, metronidazole and tinidazole, but displays improved oral absorption and longer terminal elimination half-life as compared to other antimicrobial agents in this class. It is also known as 1-(2-hydroxypropyl)-2-methyl-5-nitromidazole or 1-(2-methyl-5-nitro-1H-imidazol-1-yl) propan-2-ol and has the following chemical structure (formula I):

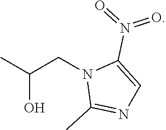

For at least the reasons described above, there is a pending need in the medical and pharmaceutical arts to develop improved treatments for diseases, such as bacterial vaginosis or trichomoniasis.

The present invention provided in this disclosure overcomes the deficiencies of existing drug compositions and formulations as described above. For example, the present invention concerns a novel soft gelatin capsule composition and formulation capable of holding low doses of secnidazole to reduce adverse effects in patients without affecting its efficacy. For example, the present invention can be vaginally administered; thereby, establishing a higher local concentration of secindazole in the vagina relative to oral administration of the same dosages but without the equivalent systemic exposures experienced from oral administration. Also, the present invention allows for the development of smaller ovules, which increases the likelihood of increased patient acceptance and compliance.

The present invention also concerns a novel soft gelatin capsule composition and formulation capable of holding large doses of secnidazole that are required for the optimal intravaginal treatment of diseases, such as bacterial vaginosis and trichomoniasis. The novel soft gelatin capsule compositions and formulations provided in this disclosure allow for high doses of secnidazole to be loaded into a single soft gelatin capsule using an excipient that retains suitable flow properties when mixed with the secnidazole and that is also compatible with latex condoms. The soft gelatin capsule composition and formulation of the present invention also possess optimal patient handling and administration properties due to the encapsulation of the drug excipient dispersions. In addition, the dose of secnidazole can be increased in the present invention to reduce the volume of capsules for easier administration vaginally, establish simpler treatment regimens (e.g., reducing the number of capsules/day), and increase patient compliance. Further, higher dosages may be more efficacious in recurrent bacterial vaginosis or with more resistant diseases or infections.

For purposes of the present invention as disclosed and described herein, the following terms and abbreviations are defined as follows.

As used herein, the term “about” as used herein, is intended to qualify the numerical values which it modifies, denoting such a value as variable within a margin of error. When no particular margin of error, such as a standard deviation to a mean value, is recited, the term “about” means plus or minus 10% of the numerical value of the number with which it is being used. Therefore, e.g., about 50% means in the range of 45%-55% a.

As used herein, the term “patient” and “subject” are interchangeable and can be taken to mean any living organism that can be treated with compounds/compositions of the present invention. As such, the terms “patient” and “subject” can include, but is not limited to, any non-human mammal, primate or human. In some embodiments, the “patient” or “subject” is an adult, child, infant, or fetus. In some embodiments, the term “patient” or “subject” is a human.

In some embodiments, the term “patient” or “subject” is a mammal, such as mice, rats, other rodents, rabbits, dogs, cats, swine, cattle, sheep, horses, primates, or humans.

In some embodiments, the terms “adjunctive administration” and “adjunctively” can be used interchangeably and refer to simultaneous administration of more than one compound in the same dosage form, simultaneous administration in separate dosage forms, and separate administration of more than one compound as part of a single therapeutic regimen.

In some embodiments, the term “daily dose amount” refers to the amount of an active agent per day that is administered or prescribed to a patient. This amount can be administered in multiple unit doses or in a single unit dose, in a single time during the day or at multiple times during the day.

In each of the embodiments disclosed herein, the compounds, pharmaceutical compositions, and methods can be utilized with or on a subject in need of such treatment, which can also be referred to as “in need thereof.” As used herein, the phrase “in need thereof” means that the subject has been identified as having a need for the particular method or treatment or that the treatment has been given to the subject for that particular purpose.

The terms “treat,” “treating,” and “treatment” are meant to include alleviating or abrogating a disease/disorder or one or more of the symptoms associated with a disease/disorder; or alleviating or eradicating the cause(s) of the disease/disorder itself. As used herein, reference to “treatment” of a disease/disorder is intended to include prevention. The terms “prevent,” “preventing,” and “prevention” refer to a method of delaying or precluding the onset of a disease/disorder; and/or its attendant symptoms, barring a subject from acquiring a disease/disorder or reducing a subject's risk of acquiring a disease/disorder. The term “treating” can be taken to mean prophylaxis of a specific disorder, disease or condition, alleviation of the symptoms associated with a specific disorder, disease or condition and/or prevention of the symptoms associated with a specific disorder, disease or condition. In some embodiments, the term refers to slowing the progression of the disorder, disease or condition or alleviating the symptoms associated with the specific disorder, disease or condition. In some embodiments, the term refers to alleviating the symptoms associated with the specific disorder, disease or condition. In some embodiments, the term refers to alleviating the symptoms associated with the specific disorder, disease or condition. In some embodiments, the term refers to restoring function which was impaired or lost due to a specific disorder, disorder or condition.

In some embodiments, the term “therapeutic” means an agent utilized to treat, combat, ameliorate or prevent an unwanted disease, condition or disorder of a patient.

In some embodiments, the term “administering” when used in conjunction with a therapeutic means to administer a therapeutic directly or indirectly into or onto a target tissue or to a patient whereby the therapeutic locally or systemically affects the tissue or the patient. “Administering” a composition can be accomplished by oral administration, injection, infusion, inhalation, absorption or by any method in combination with other known techniques. “Administering” can include the act of self-administration or administration by another person such as by a health care provider.

In some embodiments, the term “pharmaceutical composition” means a composition including at least one or more active ingredients, whereby the composition is amenable to investigation for a specified, efficacious outcome in a mammal (for example, without limitation, a human). Those of ordinary skill in the art will understand and appreciate the techniques appropriate for determining whether the active ingredients of the pharmaceutical composition have a desired efficacious outcome based upon the needs of the artisan.

In some embodiments, the term “soft gelatin capsule” shall mean a gelatin-based shell surrounding a liquid or solid fill. The soft gelatin capsules for use in the compositions described herein can be made up of a combination of gelatin, water, an opacifier and a plasticizer such as glycerin or sorbitol. The soft gelatin capsule of the present invention will be filled with an effective amount of secnidazole and a carrier such as an excipient whose physical properties, such as shape, size and consistency will facilitate its therapeutic use via intravaginal administration.

In some embodiments, the term “soft gelatin capsule composition” includes the soft gelatin capsule or any suitable encapsulation medium known in the art that is suitable for vaginal administration along with any liquid or solid fill, including the secnidazole compositions of this invention. For example, a soft gelatin capsule composition can comprise a therapeutically effective amount of secnidazole and a carrier such as an excipient as described herein along with soft gelatin capsule shell. In some embodiments, the soft gelatin capsule composition contains the therapeutically effective amount of secnidazole and an excipient and, optionally, any suitable encapsulation medium known in the art that is suitable for vaginal administration and will be suitable for dissolution and dispersion of the contents of the encapsulation medium in the vagina but will retain its integrity while being stored prior to use. In some embodiments, the soft gelatin capsule compositions disclosed herein are highly storage stable compositions having long-term storage stability while providing efficacy when used to treat the conditions disclosed herein including, but not limited to bacterial vaginosis or trichomoniasis. A soft gelatin capsule herein is a capsule that is not manufactured using preformed, 2-piece hard gelatin capsule shells but rather is manufactured as a single piece capsule by rotary die encapsulation process as described in this paragraph and paragraph [00100] in this application, and is readily understood by one skilled in the art.

The term “pharmaceutically acceptable carrier,” “pharmaceutically acceptable excipient,” “physiologically acceptable carrier,” or “physiologically acceptable excipient” refers to a pharmaceutically-acceptable material, composition, or vehicle, such as a liquid or solid filler, diluent, excipient, solvent, or encapsulating material. Each component must be “pharmaceutically acceptable” in the sense of being compatible with the other ingredients of a pharmaceutical formulation. It must also be suitable for use in contact with the tissue or organ of humans and animals without excessive toxicity, irritation, allergic response, immunogenicity, or other problems or complications, commensurate with a reasonable benefit/risk ratio. See, e.g., Remington: The Science and Practice of Pharmacy, 21st Edition; Lippincott Williams & Wilkins: Philadelphia, Pa., 2005; Handbook of Pharmaceutical Excipients, 5th Edition; Rowe et al., Eds., The Pharmaceutical Press and the American Pharmaceutical Association: 2005; and Handbook of Pharmaceutical Additives, 3rd Edition; Ash and Ash Eds., Gower Publishing Company: 2007; Pharmaceutical Preformulation and Formulation, Gibson Ed., CRC Press LLC: Boca Raton, Fla., 2004).

The terms “therapeutically effective amount” or “therapeutic dose” are used interchangeably and refer to the amount of an active agent or pharmaceutical compound or composition that elicits a clinical, biological or medicinal response in a tissue, system, animal, individual or human that is being sought by a researcher, veterinarian, medical doctor, or other clinical professional. A clinical, biological or medical response can include, for example, one or more of the following: (1) preventing a disease, condition or disorder in an individual that can be predisposed to the disease, condition or disorder but does not yet experience or display pathology or symptoms of the disease, condition or disorder, (2) inhibiting a disease, condition or disorder in an individual that is experiencing or displaying the pathology or symptoms of the disease, condition or disorder or arresting further development of the pathology and/or symptoms of the disease, condition or disorder, and (3) ameliorating a disease, condition or disorder in an individual that is experiencing or exhibiting the pathology or symptoms of the disease, condition or disorder or reversing the pathology and/or symptoms experience or exhibited by the individual.

In accordance with an aspect of the present invention, a compound of formula I which is secnidazole, a prodrug thereof, a hydrate thereof, a solvate thereof, a polymorph thereof, or a pharmaceutically acceptable salt thereof.

The term “pharmaceutically acceptable salt” for the purpose of present invention is meant to indicate, without any limitation, those salts which are within the scope of sound medical judgment, suitable for use in contact with the tissues of a patient, without undue toxicity, irritation, allergic response and the like, and are commensurate with a reasonable benefit/risk ratio. Pharmaceutically acceptable salts suitable for the present invention are well-known in the art and described in, for instance, Berge et al., “Pharmaceutical Salts,” J. Pharm. Sci., 66(1):1-19 (1977). For example, in one embodiment, a pharmaceutically acceptable salt of the present invention can be any acid addition salt, preferably a pharmaceutically acceptable acid addition salt, including, but not limited to, halogenic acid salts such as hydrobromic, hydrochloric, hydrofloric and hydroiodic acid salt; an inorganic acid salt, such as, nitric, perchloric, sulfuric and phosphoric acid salt; an organic acid salt, such as, sulfonic acid salts (such as methanesulfonic, trifluoromethan sulfonic, ethanesulfonic, benzenesulfonic or p-toluenesufonic), acetic, malic, fumaric, succinic, citric, benzonic gluconic, lactic, mandelic, mucic, pamoic, pantothenic, oxalic and maleic acid salts; or an amino acid salt such as aspartic or glutamic acid salt. The acid addition salt can be a mono- or di-acid addition salt, such as a di-hydrohalogic, di-sulfuric, di-phosphoric or di-organic acid salt. In all cases, the acid addition salt is used as an achiral reagent which is not selected on the basis of any expected or known preference for the interaction with or precipitation of a specific optical isomer of the products of this disclosure.

The term “prodrug” refers to a functional derivative of the compound as disclosed herein and is readily convertible into the parent compound in vivo. Prodrugs are often useful because, in some situations, they can be easier to administer than the parent compound. They can, for instance, be bioavailable by oral administration whereas the parent compound is not. The prodrug can also have enhanced solubility in pharmaceutical compositions over the parent compound. A prodrug can be converted into the parent drug by various mechanisms, including enzymatic processes and metabolic hydrolysis (see, e.g., Rautio et al., “The Expanding Role of Prodrugs in Contemporary Drug Design and Development,” Nature Reviews Drug Discovery 17:559-587 (2018); Clas et al., “Chemistry-enabled Drug Delivery (prodrugs): Recent Progress and Challenges,” Drug Discov. Today 19:79-87 (2014); Rautio, J. Prodrugs and Targeted Delivery, Wiley-VCH Verlag GmbH & Co. KGaA (2011); Stella, V. J. et al. Prodrugs: Challenges and Rewards Vol. 1-2, Springer & AAPS Press (2007); Rautio, J. et al., “Prodrugs: design and clinical applications,” Nat. Rev. Drug Discov. 7,255-270 (2008)).

As used herein, the term “solvate” refers to a complex that is formed by combining the molecules or ions of a solvent with a compound. In other words, the term “solvate” refers to the complex that is obtained by the process of solvation wherein solvent and solute molecules reorganize into solvation complexes. The term “solvates” includes crystal forms containing either stoichiometric or nonstoichiometric amounts of a solvent.

As used herein, the term “hydrate” refers to a compound that absorbs water molecules from its environment and includes them as part of its structure. In some instances, the hydrates of organic compounds are such that the water chemically reacts with the organic compound. In some instances, the term “hydrate” includes a solvate where the incorporated solvent is water.

As used herein, the term “polymorph” refers to crystalline polymorphic forms and amorphous polymorphic forms of a compound. Crystalline polymorphs have different arrangements and/or conformations of the molecules in crystal lattice. Amorphous polymorphs consist of disordered arrangements of molecules that do not possess a distinguishable crystal lattice.

In some embodiments, in the soft gelatin capsule compositions, the pharmaceutically acceptable carrier is an excipient comprising a fatty acid derivative, which can be is a derivative of a mono-unsaturated fatty acid. In some embodiments, the excipient comprises a polyoxylglyceride of a mono-unsaturated fatty acid. In some embodiments, the polyoxylglyceride in the excipient is selected from the group consisting of monoglycerides oleic acid (C18:1), diglycerides oleic acid (C18:1), triglycerides oleic acid (C18:1), mono-polyethylene glycol-6 esters of oleic acid (C18:1), di-polyethylene glycol-6 esters of oleic acid (C18:1), and any combination thereof.

In some embodiments, the fatty acid derivative in the excipient is a derivative of a saturated fatty acid.

In some embodiments, the fatty acid derivative can include a derivative of saturated fatty acid, a derivative of a mono-unsaturated fatty acid, or a combination thereof. For example, the excipient can comprise oleoyl polyoxyl-6 glycerides; triglycerides of caprylic and capric acids; or a combination thereof.

As used herein, the term “monounsaturated fatty acid,” or “mono-unsaturated fatty acid,” refers to a fatty acid having one double or triple bond in the hydrocarbon chain. Examples of monounsaturated fatty acids include, but are not limited to, octenoic acid (C8:1), decenoic acid (C10:1), oleic acid (C18:1), lauroleic acid (C12:1), eicosenoic acid (C20:1) and nervonic acid (C24:1), or any combination thereof. By way of example, C18:1 indicates oleic acid has eighteen carbon atoms and one double bond in its hydrocarbon chain. The number of carbon atoms and double bonds in the hydrocarbon chain of other example unsaturated fatty acids is denoted in analogous fashion herein.

As used herein, the term “glyceride” refers to an ester resulting from glycerol and at least one fatty acid. In some embodiments, a glyceride can include mono-, di- and triglycerides of one fatty acid, or any combination thereof. In some embodiments, a glyceride can include mono-, di- and triglycerides of two or more different fatty acids, or any combination thereof. For example, Oleoyl polyoxyl-6 glycerides is available from Gattefosse under tradename of LABRAFIL® M1944CS, which consists of mono-, di- and triglycerides and PEG-6 (MW 300) mono- and diesters of oleic (C18:1) acid.

As another example, medium chain triglycerides are available from Gattefosse under tradename of LABRAFAC® lipophil WL 1349, which consists of medium-chain triglycerides of caprylic (C8) and capric (decanoic, C10) acids. Both caprylic (C8) and capric (decanoic, C10) acids are saturated fatty acids. As an example, LABRAFAC® lipophil WL 1349 is a saturated fatty acid derivative.

In another aspect of the present invention, soft gelatin capsules or ovules can be administered vaginally with more consistency and with greater patient acceptance than gels, creams, or suppositories,

In accordance with another aspect of the present invention, a soft gelatin capsule compositions for vaginal administration including (a) secnidazole, a prodrug thereof, a hydrate thereof, a solvate thereof, a polymorph thereof, a pharmaceutically acceptable salt thereof, or a combination thereof, and (b) at least one pharmaceutically acceptable carrier.

In addition to pharmaceutically acceptable salts, the soft gelatin capsule compositions for vaginal administration can contain hydrates and solvates of secnidazole as the active ingredient. For example, the secnidazole hemi-hydrate has been found to be particularly useful for inclusion in the pharmaceutical composition.

In certain embodiments, the therapeutically effective amount of secnidazole, the prodrug thereof, the hydrate thereof, the solvate thereof, the polymorph thereof, or the pharmaceutically acceptable salt thereof in the soft gelatin capsule compositions for vaginal administration is from about 0.1 mg to about 1,500 mg.

In certain embodiment, soft gelatin capsule compositions for vaginal administration include the therapeutically effective amount of secnidazole is from 1 mg to about 500 mg or other ranges thereinbetween (e.g., from 1 mg to about 499 mg; from 1 mg to about 450 mg; from 1 mg to about 400 mg; from 1 mg to about 300 mg; from 1 mg to about 200 mg; from 1 mg to about 100 mg).

In yet another embodiment, soft gelatin capsule compositions for vaginal administration includes about 35 mg, about 50 mg, about 75 mg, about 100 mg, about 150 mg, about 200 mg, about 300 mg, about 400 mg, about 450 mg, about 490 mg, or about 500 mg of secnidazole, a pharmaceutically acceptable secnidazole salt, or a pharmaceutically acceptable secnidazole hydrate or solvate.

In certain embodiment, soft gelatin capsule compositions for vaginal administration include the therapeutically effective amount of secnidazole is from 1,000 mg to about 1,500 mg or other ranges thereinbetween (e.g., from 1,001 mg to about 1,500 mg; from 1,100 mg to about 1,500 mg; from 1,200 mg to about 1,500 mg; from 1,300 mg to about 1,500 mg; from 1,400 mg to about 1,500 mg). In yet another embodiment, the pharmaceutical composition includes about 1,000 mg, about 1,100 mg, about 1,200 mg, about 1,300 mg, about 1,400 mg, or about 1,500 mg of secnidazole, a pharmaceutically acceptable secnidazole salt, or a pharmaceutically acceptable secnidazole hydrate or solvate.

In another aspect described of the present invention, soft gelatin capsule compositions for vaginal administration of secnidazole and a therapeutically effective amount of secnidazole dispersed in a fill composition suitable for encapsulation within soft gelatin capsules. The fill composition includes secnidazole, pharmaceutically acceptable secnidazole salts, or pharmaceutically acceptable secnidazole hydrates or solvates. Further embodiments are directed to soft gelatin capsule compositions for vaginal administration of secnidazole comprising a therapeutically effective amount of secnidazole dispersed in a carrier such as an excipient comprising a derivative of a fatty acid such as a mono-unsaturated fatty acid, with or without in the inclusion of viscosity modifying agents.

The soft gelatin capsule compositions of the present invention can also contain one pharmaceutically acceptable carrier selected from additives, such as stabilizers (e.g., antioxidants and other types of preservatives), polymorphic transition accelerators (e.g., tristearin), viscosity modifying agents, biocompatible polymers, surfactants, dispersants, water absorbents and the like. The use of biocompatible polymers, surfactants and water absorbents are described in U.S. Pat. No. 4,765,978, the disclosure of which is hereby incorporated by reference. The concentration of these additives can vary according to the particular additive used and the desired result sought. The use of the kind and concentration of additives are well within the ability of the skilled artisan.

Further embodiments include the excipient is a derivative of a mono-unsaturated fatty acid in some embodiments. In some embodiments, the excipient is a polyoxylglyceride excipient. Further embodiments include the polyoxylglyceride excipient comprises monoglycerides oleic acid (C18:1), diglycerides oleic acid (C18:1), triglycerides oleic acid (C18:1), mono-polyethylene glycol-6 esters of oleic acid (C18:1), di-polyethylene glycol-6 esters of oleic acid (C18:1), or any combination thereof. A further embodiment includes Labrafil M1944 CS (oleoyl polyoxyl-6 glycerides), which contains mono-, di- and triglycerides, and PEG-6 (MW 300) mono- and diesters of oleic (18:1) acid.

Further embodiments include the viscosity modifying agent excipient is a polyethylene glycol excipient. Further embodiments include polyethylene glycol excipient is one of polymer molecular weight of 3350. Polyethylene glycols of other molecular weights can also be used in addition to other viscosity modifying agents. Secnidazole compatibility has been previously assessed with PEGs (Mw 400 and 1500). Reduction in the secnidazole fill content to achieve lower dosage strength compositions can result in lower fill viscosities that can be adjusted by using higher molecular weight polyethylene glycols depending on the degree to which the secnidazole fill content is reduced to allow for reproducible encapsulation and for consistent administration to patients.

Another aspect of the present invention relates to a soft gelatin capsule composition for vaginal administration of secnidazole comprising a therapeutically effective amount of secnidazole dispersed in a pharmaceutically acceptable carrier, wherein the therapeutically effective amount of secnidazole is in a range of from about 1 mg to about 500 mg or other ranges thereinbetween (e.g., from about 1 mg to about 499 mg; from about 1 mg to about 450 mg; from about 1 mg to about 400 mg; from about 1 mg to about 300 mg; from about 1 mg to about 200 mg; from about 1 mg to about 100 mg).

In some embodiments, in a soft gelatin capsule composition for vaginal administration, the therapeutically effective amount of secnidazole is about 35 mg.

In some embodiments, in a soft gelatin capsule composition for vaginal administration, the therapeutically effective amount of secnidazole is about 50 mg.

In some embodiments, in a soft gelatin capsule composition for vaginal administration, the therapeutically effective amount of secnidazole is about 75 mg.

In some embodiments, in a soft gelatin capsule composition for vaginal administration, the therapeutically effective amount of secnidazole is about 100 mg.

In some embodiments, in a soft gelatin capsule composition for vaginal administration, the therapeutically effective amount of secnidazole is about 150 mg.

In some embodiments, in a soft gelatin capsule composition for vaginal administration, the therapeutically effective amount of secnidazole is about 200 mg;

In some embodiments, in a soft gelatin capsule composition for vaginal administration, the therapeutically effective amount of secnidazole is about 300 mg.

In some embodiments, in a soft gelatin capsule composition for vaginal administration, the therapeutically effective amount of secnidazole is about 400 mg.

In some embodiments, in a soft gelatin capsule composition for vaginal administration, the therapeutically effective amount of secnidazole is about 450 mg.

In some embodiments, in a soft gelatin capsule composition for vaginal administration, the therapeutically effective amount of secnidazole is about 490 mg.

In some embodiments, in a soft gelatin capsule composition for vaginal administration, the therapeutically effective amount of secnidazole is about 499 mg or 500 mg.

In some embodiments, soft gelatin capsule composition for vaginal administration of secnidazole comprising a therapeutically effective amount of secnidazole dispersed in a pharmaceutically acceptable carrier, wherein the therapeutically effective amount of secnidazole is in a range of from about 1 mg to about 500 mg or other ranges thereinbetween (e.g., from about 1 mg to about 499 mg; from about 1 mg to about 450 mg; from about 1 mg to about 400 mg; from about 1 mg to about 300 mg; from about 1 mg to about 200 mg; from about 1 mg to about 100 mg), wherein the pharmaceutically acceptable carrier is a derivative of a fatty acid such as a mono-unsaturated fatty acid. In another embodiment, the mono-unsaturated fatty acid-based excipient is a polyoxylglyceride excipient.

In preferred embodiment, the polyoxylglyceride excipient comprises monoglycerides oleic acid (C18:1), diglycerides oleic acid (C18:1), triglycerides oleic acid (C18:1), mono-polyethylene glycol-6 esters of oleic acid (C18:1), di-polyethylene glycol-6 esters of oleic acid (C18:1), or any combination thereof.

In more preferred embodiment, the excipient is an oleoyl polyoxyl-6 glyceride (such as Labrafil M1944 CS), a medium chain triglyceride (such as Labrafac Lipophile WL1349), or a combination thereof.

In another embodiment, soft gelatin capsule compositions for vaginal administration of secnidazole comprising a therapeutically effective amount of secnidazole dispersed in a pharmaceutically acceptable carrier, wherein the therapeutically effective amount of secnidazole is in a range of from about 1,000 mg to about 1,500 mg or other ranges thereinbetween (e.g., from about 1,001 mg to about 1,500 mg; from about 1,100 mg to about 1,500 mg; from about 1,200 mg to about 1,500 mg; from about 1,300 mg to about 1,500 mg; from about 1,400 mg to about 1,500 mg), wherein the pharmaceutically acceptable carrier is a derivative of a fatty acid such as a mono-unsaturated fatty acid.

In some embodiments, in a soft gelatin capsule composition for vaginal administration, the therapeutically effective amount of secnidazole is about 1,100 mg.

In some embodiments, in a soft gelatin capsule composition for vaginal administration, the therapeutically effective amount of secnidazole is about 1,200 mg.

In some embodiments, in a soft gelatin capsule composition for vaginal administration, the therapeutically effective amount of secnidazole is about 1,300 mg.

In some embodiments, in a soft gelatin capsule composition for vaginal administration, the therapeutically effective amount of secnidazole is about 1,400 mg.

In some embodiments, in a soft gelatin capsule composition for vaginal administration, the therapeutically effective amount of secnidazole is about 1,500 mg.

In another embodiment, soft gelatin capsule composition for vaginal administration of secnidazole comprising a therapeutically effective amount of secnidazole dispersed in a pharmaceutically acceptable carrier, wherein the therapeutically effective amount of secnidazole is from about 1,000 mg to about 1,500 mg or other ranges thereinbetween (e.g., from about 1,001 mg to about 1,500 mg; from about 1,100 mg to about 1,500 mg; from about 1,200 mg to about 1,500 mg; from about 1,300 mg to about 1,500 mg; from about 1,400 mg to about 1,500 mg), wherein the pharmaceutically acceptable carrier is a derivative of a fatty acid such as a mono-unsaturated fatty acid. In some embodiments, the excipient is a derivative of a mono-unsaturated fatty acid, which is a polyoxylglyceride excipient.

In preferred embodiment, the polyoxylglyceride excipient comprises monoglycerides oleic acid (C18:1), diglycerides oleic acid (C18:1), triglycerides oleic acid (C18:1), mono-polyethylene glycol-6 esters of oleic acid (C18:1), di-polyethylene glycol-6 esters of oleic acid (C18:1), or any combination thereof.

In more preferred embodiment, the excipient is an oleoyl polyoxyl-6 glyceride (such as Labrafil M1944 CS), a medium chain triglyceride (such as Labrafac Lipophile WL1349), or a combination thereof.

Another aspect of the present invention relates to a method of manufacturing the pharmaceutical composition by a process known as encapsulation using the rotary die encapsulation process. Manufacturing properties, such as the viscosity of the secnidazole fill material, when the fill material is in a fluid state during processing, will also determine the minimum amount of fill composition that is needed to disperse, mold and package a soft gelatin capsule having a given amount of secnidazole. Such a parameter is not critical to the present invention and can be determined in the course of routine optimization of the manufacturing process. The soft gelatin capsule compositions will be in the range of 0.5 to 10 g, preferably 1 to 5 g, and most preferably 1 to 3 g.

The soft gelatin capsule composition can comprise secnidazole in a range of from about 0.1% to about 60% by weight of the total composition. Preferably, the soft gelatin capsule composition can comprise secnidazole in a range of about 20% to about 40%, more preferably, in a range of from about 30% to about 40%, and most preferably, in a range of from about 35% to about 40%. In some embodiments, the soft gelatin capsule compositions comprise about 37% to about 38% of secnidazole.

The soft gelatin capsule compositions of the present invention can be produced in a process known as encapsulation using the rotary die encapsulation process. The encapsulation process can be described as a form/fill/seal process. In some embodiments, two flat ribbons of shell material are manufactured on the machine and brought together on a twin set of rotating dies. The dies contain recesses in the desired size and shape, which cut out the ribbons into a two-dimensional shape, and form a seal around the outside. At the same time a pump delivers a precise dose of fill material (i.e., one or more excipients and a therapeutically effective amount of secnidazole) through a nozzle incorporated into a filling wedge whose tip sits between the two ribbons in between two die pockets at the point of cut out. The wedge is heated to facilitate the sealing process. The wedge injection causes the two flat ribbons to expand into the die pockets, giving rise to the three-dimensional finished product. After encapsulation, the soft gelatin capsule compositions are dried for about two days to about two weeks.

The contents of soft gelatin capsule compositions of the present invention can be solid or liquid at room temperature, and preferably have a flow point in the range of 30° C. to 40° C., more preferably 30° C. to 37° C. The flow point is visually determined based upon heating a sample from 25° C. at a rate of 2° C./minute and observing the temperature at which rapid flow of the sample occurs. This measurement is conveniently carried out using a microscope equipped with a video camera having on-screen digital monitoring of the temperature. In some embodiments the contents of the soft gelatin capsule compositions can be liquid at room temperature

The soft gelatin capsule compositions described herein can be prepared, packaged, or sold in bulk, as a single unit dose or as multiple unit doses and can be administered in the conventional manner by any route where they are active.

In another aspect of the present invention, other capsule shapes in addition to the oval capsules provided in the examples can also be used in the practice of the present invention.

In some embodiments, soft gelatin capsules for vaginal administration can be prepared in a variety of geometries. For example, vaginal soft gelatin capsules can be shaped as an oval, a tear drop, a cone with frustoconical end, a cylinder, a cylinder with larger “cap” portion, a round, a bullet, a tampon, a globular, an oviform or other shapes suitable for insertion into the vagina. Vaginal soft gelatin capsules in accordance with various embodiments can or cannot be used in connection with an applicator.

Another aspect of the present invention relates to a method for treating bacterial vaginosis in a subject in need thereof. The method includes selecting the subject in need of treatment for bacterial vaginosis and administering, to the subject, a pharmaceutical composition including a therapeutically effective amount of secnidazole, pharmaceutically acceptable secnidazole salt, or pharmaceutically acceptable secnidazole hydrate, solvate, prodrug, or polymorph thereof. The amount of secnidazole or a pharmaceutically acceptable salt thereof in the pharmaceutical composition is in a range of from about 1 mg to about 500 mg or other ranges thereinbetween (e.g., from about 1 mg to about 499 mg; from about 1 mg to about 450 mg; from about 1 mg to about 400 mg; from about 1 mg to about 300 mg; from about 1 mg to about 200 mg; from about 1 mg to about 100 mg) and the pharmaceutical composition is in the form of a vaginal soft gelatin capsule.

In some embodiments, a method for treating bacterial vaginosis in a subject in need thereof comprises selecting the subject in need of treatment for bacterial vaginosis and administering to the subject soft gelatin capsule composition, wherein the therapeutically effective amount of secnidazole is about 35 mg.

In some embodiments, a method for treating bacterial vaginosis in a subject in need thereof comprises selecting the subject in need of treatment for bacterial vaginosis and administering to the subject soft gelatin capsule composition, wherein the therapeutically effective amount of secnidazole is about 50 mg.

In some embodiments, a method for treating bacterial vaginosis in a subject in need thereof comprises selecting the subject in need of treatment for bacterial vaginosis and administering to the subject soft gelatin capsule composition, wherein the therapeutically effective amount of secnidazole is about 75 mg.

In some embodiments, a method for treating bacterial vaginosis in a subject in need thereof, selecting the subject in need of treatment for bacterial vaginosis and administering to the subject soft gelatin capsule composition, wherein the therapeutically effective amount of secnidazole is about 100 mg.

In some embodiments, a method for treating bacterial vaginosis in a subject in need thereof comprises selecting the subject in need of treatment for bacterial vaginosis and administering to the subject soft gelatin capsule composition, wherein the therapeutically effective amount of secnidazole is about 150 mg.

In some embodiments, a method for treating bacterial vaginosis in a subject in need thereof comprises selecting the subject in need of treatment for bacterial vaginosis and administering to the subject soft gelatin capsule composition, wherein the therapeutically effective amount of secnidazole is about 200 mg;

In some embodiments, a method for treating bacterial vaginosis in a subject in need thereof comprises selecting the subject in need of treatment for bacterial vaginosis and administering to the subject soft gelatin capsule composition, wherein the therapeutically effective amount of secnidazole is about 300 mg.

In some embodiments, a method for treating bacterial vaginosis in a subject in need thereof comprises selecting the subject in need of treatment for bacterial vaginosis and administering to the subject soft gelatin capsule composition, wherein the therapeutically effective amount of secnidazole is about 400 mg.

In some embodiments, a method for treating bacterial vaginosis in a subject in need thereof comprises selecting the subject in need of treatment for bacterial vaginosis and administering to the subject soft gelatin capsule composition, wherein the therapeutically effective amount of secnidazole is about 450 mg or 500 mg.

Another aspect of the present invention relates to a method for further treating bacterial vaginosis in a subject in need thereof. The method includes selecting the subject in need of treatment for bacterial vaginosis and administering, to the subject, a pharmaceutical composition including a therapeutically effective amount of secnidazole, pharmaceutically acceptable secnidazole salt, or pharmaceutically acceptable secnidazole hydrate, solvate, prodrug, or polymorph thereof. The amount of secnidazole or a pharmaceutically acceptable salt thereof in the pharmaceutical composition is in a range of from about 1,000 mg to about 1,500 mg or other ranges thereinbetween (e.g., from about 1,001 mg to about 1,500 mg; from about 1,100 mg to about 1,500 mg; from about 1,200 mg to about 1,500 mg; from about 1,300 mg to about 1,500 mg; from about 1,400 mg to about 1,500 mg) and the pharmaceutical composition is in the form of a vaginal soft gel capsule.

In some embodiments, a method for treating bacterial vaginosis in a subject in need thereof comprises selecting the subject in need of treatment for bacterial vaginosis and administering to the subject soft gelatin capsule composition, wherein the therapeutically effective amount of secnidazole is about 1,100 mg.

In some embodiments, a method for treating bacterial vaginosis in a subject in need thereof comprises selecting the subject in need of treatment for bacterial vaginosis and administering to the subject soft gelatin capsule composition, wherein the therapeutically effective amount of secnidazole is about 1,200 mg

In some embodiments, a method for treating bacterial vaginosis in a subject in need thereof comprises selecting the subject in need of treatment for bacterial vaginosis and administering to the subject soft gelatin capsule composition, wherein the therapeutically effective amount of secnidazole is about 1,300 mg.

In some embodiments, a method for treating bacterial vaginosis in a subject in need thereof comprises selecting the subject in need of treatment for bacterial vaginosis and administering to the subject soft gelatin capsule composition, wherein the therapeutically effective amount of secnidazole is about 1,400 mg.

In some embodiments, a method for treating bacterial vaginosis in a subject in need thereof comprises selecting the subject in need of treatment for bacterial vaginosis and administering to the subject soft gelatin capsule composition, wherein the therapeutically effective amount of secnidazole is about 1,500 mg.

In some embodiments, the soft gelatin compositions described herein can be administered once a day for a period of 1, 2, 3, 4, 5, 6, 7 days or longer. In some embodiments, the soft gelatin compositions described herein can be administered once, twice, or three times per day for a period of 1, 2, 3, 4, 5, 6, 7 days or longer. In some embodiments, the administering the soft gel capsule compositions at the doses and frequency of administration described herein results in treatment of the bacterial vaginosis in the patient.

Some embodiments are directed to methods of treating a condition in a patient in need thereof, comprising administering intravaginally to the patient a soft gelatin capsule composition of secnidazole, comprising a therapeutically effective amount of secnidazole dispersed in an excipient. In some embodiments, the condition is a vaginal infection. In some embodiments, the vaginal infection is caused by an overgrowth of bacteria such as Gardenia vaginalis. In some embodiments, the condition is an infection in the vagina caused by an anaerobic bacteria or a parasite. In some embodiments, the condition is an infection in the vagina caused by gram negative bacteria. In some embodiments, the condition is bacterial vaginosis, trichomoniasis, or any combination thereof. In some embodiments, the condition is an imbalance of the naturally occurring bacteria in the vagina.

In some embodiments, therapeutically effective amounts, daily doses, or single unit doses of the secnidazole compositions described herein can be administered once per day or multiple times per day, such as 1 to 5 doses, twice per day or three times per day. In certain embodiments, one soft gelatin capsule composition comprising a therapeutically effective amount of the secnidazole composition is administered once to said patient and the bacterial vaginosis is treated.

Embodiments are also directed to a dosage regimen for treating bacterial vaginosis in patient comprising administering secnidazole, such as secnidazole compound, to treat the conditions disclosed herein. For example, in some embodiments, the methods described herein can comprise a dosage regimen that can include a plurality of daily doses having an equal amount of secnidazole compound as the initial dose in one or more unit doses. In other embodiments, the dosage regimen can include an initial dose of secnidazole, such as secnidazole compound in one or more unit doses, then a plurality of daily doses having a lower amount of secnidazole compound as the initial dose in one or more unit doses. The dosage regimen can administer an initial dose followed by one or more maintenance doses. The plurality of doses following the administering of an initial dose can be maintenance doses.

The selection of the specific dose regimen can be adjusted or titrated by the clinician according to methods known to the clinician in order to obtain the optimal clinical response. The amount of secnidazole compound to be administered can be an amount that is therapeutically effective. The dosage to be administered can depend on the characteristics of the subject being treated, e.g., the particular animal or human subject treated, age, weight, body mass index, body surface area, health, types of concurrent treatment, if any, and frequency of treatments, and can be easily determined by one of skill in the art (e.g., by the clinician).

Another aspect of the present invention relates to a method for treating trichomoniasis in a subject in need thereof. The method includes selecting the subject in need of treatment for trichomoniasis and administering, to the subject, a pharmaceutical composition including a therapeutically effective amount of secnidazole, or pharmaceutically acceptable salt, prodrug, solvate, hydrate, or polymorph thereof. The amount of secnidazole or a pharmaceutically acceptable salt thereof in the pharmaceutical composition is in a range of from about 1 mg to about 500 mg or other ranges thereinbetween (e.g., from about 1 mg to about 499 mg; from about 1 mg to about 450 mg; from about 1 mg to about 400 mg; from about 1 mg to about 300 mg; from about 1 mg to about 200 mg; from about 1 mg to about 100 mg) and the pharmaceutical composition is in the form of a vaginal soft gel capsule.

In some embodiments, a method for treating trichomoniasis in a subject in need thereof comprises selecting the subject in need of treatment for bacterial vaginosis and administering to the subject soft gelatin capsule composition, wherein the therapeutically effective amount of secnidazole is about 35 mg.

In some embodiments, a method for treating trichomoniasis in a subject in need thereof comprises selecting the subject in need of treatment for bacterial vaginosis and administering to the subject soft gelatin capsule composition, wherein the therapeutically effective amount of secnidazole is about 50 mg.

In some embodiments, a method for treating trichomoniasis in a subject in need thereof comprises selecting the subject in need of treatment for bacterial vaginosis and administering to the subject soft gelatin capsule composition, wherein the therapeutically effective amount of secnidazole is about 75 mg.

In some embodiments, a method for treating trichomoniasis in a subject in need thereof comprises selecting the subject in need of treatment for bacterial vaginosis and administering to the subject soft gelatin capsule composition, wherein the therapeutically effective amount of secnidazole is about 100 mg.

In some embodiments, a method for treating trichomoniasis in a subject in need thereof comprises selecting the subject in need of treatment for bacterial vaginosis and administering to the subject soft gelatin capsule composition, wherein the therapeutically effective amount of secnidazole is about 150 mg.

In some embodiments, a method for treating trichomoniasis in a subject in need thereof comprises selecting the subject in need of treatment for bacterial vaginosis and administering to the subject soft gelatin capsule composition, wherein the therapeutically effective amount of secnidazole is about 200 mg;

In some embodiments, a method for treating trichomoniasis in a subject in need thereof comprises selecting the subject in need of treatment for bacterial vaginosis and administering to the subject soft gelatin capsule composition, wherein the therapeutically effective amount of secnidazole is about 300 mg.

In some embodiments, a method for treating trichomoniasis in a subject in need thereof comprises selecting the subject in need of treatment for bacterial vaginosis and administering to the subject soft gelatin capsule composition, wherein the therapeutically effective amount of secnidazole is about 400 mg.

In some embodiments, a method for treating trichomoniasis in a subject in need thereof comprises selecting the subject in need of treatment for bacterial vaginosis and administering to the subject soft gelatin capsule composition, wherein the therapeutically effective amount of secnidazole is about 450 mg or 500 mg.

Another aspect of the present invention relates to a method for further treating trichomoniasis in a subject in need thereof. The method includes selecting the subject in need of treatment for trichomoniasis and administering, to the subject, a pharmaceutical composition including a therapeutically effective amount of secnidazole, or pharmaceutically acceptable salt, prodrug, solvate, hydrate, or polymorph thereof. The amount of secnidazole or a pharmaceutically acceptable salt thereof in the pharmaceutical composition is in a range of from about 1,000 mg to about 1,500 mg or other ranges thereinbetween (e.g., from about 1,001 mg to about 1,500 mg; from about 1,100 mg to about 1,500 mg; from about 1,200 mg to about 1,500 mg; from about 1,300 mg to about 1,500 mg; from about 1,400 mg to about 1,500 mg) and the pharmaceutical composition is in the form of a vaginal soft gelatin capsule.

In some embodiments, a method for treating trichomoniasis in a subject in need thereof comprises selecting the subject in need of treatment for bacterial vaginosis and administering to the subject soft gelatin capsule composition, wherein the therapeutically effective amount of secnidazole is about 1,100 mg.

In some embodiments, a method for treating trichomoniasis in a subject in need thereof comprises selecting the subject in need of treatment for bacterial vaginosis and administering to the subject soft gelatin capsule composition, wherein the therapeutically effective amount of secnidazole is about 1,200 mg.

In some embodiments, a method for treating trichomoniasis in a subject in need thereof comprises selecting the subject in need of treatment for bacterial vaginosis and administering to the subject soft gelatin capsule composition, wherein the therapeutically effective amount of secnidazole is about 1,300 mg.

In some embodiments, a method for treating trichomoniasis in a subject in need thereof comprises selecting the subject in need of treatment for bacterial vaginosis and administering to the subject soft gelatin capsule composition, wherein the therapeutically effective amount of secnidazole is about 1,400 mg.

In some embodiments, a method for treating trichomoniasis in a subject in need thereof comprises selecting the subject in need of treatment for bacterial vaginosis and administering to the subject soft gelatin capsule composition, wherein the therapeutically effective amount of secnidazole is about 1,500 mg.

In another aspect, therapeutically effective amounts of secnidazole can be delivered vaginally depending on the indication intended for treatment and that soft gelatin ovules can be administered vaginally with more consistency and with greater patient acceptance than gels, creams, or suppositories, soft gelatin capsule compositions for vaginal administration of secnidazole and a therapeutically effective amount of secnidazole dispersed in a fill composition suitable for encapsulation within soft gelatin capsules represent advantages over the currently approved products for these indications. In addition, secnidazole can treat trichomoniasis and vaginal administration of secnidazole can produce a better or equivalent treatment outcome than orally administered secnidazole or vaginally administered metronidazole.

The following example illustrates aspects of the present invention, and is set forth to assist in understanding the present invention. This example should not be construed as specifically limiting the present invention described and claimed herein. Variations of the present invention, including the substitution of all equivalents now known or later developed, which would be within the purview of those skilled in the art, and changes in composition, formulation or minor changes in experimental design, are considered to fall within the scope of the present invention and appended claims.

### Examples

The following Examples are intended to be illustrative and are in no way intended to limit the scope of the present invention.

Two approaches can be used to formulate the soft gelatin capsule fill compositions and formulations disclosed in this application. In a first approach, a soft gelatin capsule fill composition is kept consistent while a fill amount encapsulated is changed to achieve the target dosage resulting in different sizes soft gelatin capsules, which can be oval-shaped, tear drop-shaped, cone with frustoconical end-shaped, cylinder-shaped, cylinder with larger “cap” portion-shaped, round-shaped, bullet-shaped, tampon-shaped, globular-shaped, oviform-shaped or in other shapes suitable for insertion into the vagina. Alternatively, in a second approach, the target soft gelatin capsule size is kept consistent while the secnidazole concentration is changed in the soft gelatin capsule fill composition.

An example of using the first approach mentioned above, a soft gelatin capsule fill composition that can be used to manufacture a vaginal soft gelatin capsule composition comprising secnidazole is provided in Table 1.

TABLE 1
Example Secnidazole Vaginal Soft
Gelatin Capsule Fill Composition

| Ingredient | w/w % |
|---|---|
| Secnidazole (as a hemi-hydrate)* | 37.70 |
| Oleoyl polyoxyl-6 glycerides** | 61.37 |
| Polysorbate 80 | 0.43 |
| Lecithin | 0.50 |

*Can also be a pharmaceutically acceptable salt, prodrug or solvate of secnidazole
**Labrafil M1944 CS

Additional soft gelatin capsule fill compositions with higher secnidazole concentrations may result in higher strength ovules (such as 1,000 mg-1,500 mg) with a size 40 oval or smaller soft gelatin capsule size.

Using the soft gelatin capsule fill composition provided in Table 1, examples of secnidazole vaginal ovule sizes as a function of secnidazole dosage amount is provided in Table 2. Secnidazole dosage amounts (other than those specified in Table 2) can be obtained by changing the target soft gelatin size. For example, increasing the soft gelatin oval capsule size can be used to achieve examples of higher target dosages of secnidazole.

TABLE 2
Various Example Secnidazole Vaginal Soft Gelatin Capsule
Sizes As A Function Of Secnidazole Dosage Amounts

| Soft | | | | | | |
|---|---|---|---|---|---|---|
| Gelatin | | | | | | |
| Capsule | | | | | | |
| Character- | 100 | 300 | 400 | 500 | 1000 | 1500 |
| istics | mg | mg | mg | mg | mg | mg |
| Fill weight | 0.27 | 0.80 | 1.06 | 1.33 | 2.65 | 3.98 |
| (g) | | | | | | |
| Fill volume | 0.28 | 0.84 | 1.11 | 1.39 | 2.78 | 4.18 |
| (cm³) | | | | | | |
| Target Oval | 5 | 16 | 20 | 30 | 60 | 65 |
| Soft Gelatin | | | | | | |
| Capsule Size | | | | | | |

Further, additional examples of soft gelatin capsule fill compositions that can be used to manufacture secnidazole vaginal soft gelatin capsules are provided in Table 3. With this approach, secnidazole capsule size would be kept constant while the amount of secnidazole in the fill composition would vary. For example, the fill composition examples provided in Tables 3 and 4 show a constant secnidazole soft gelatin capsule size of Size 30 Oval.

TABLE 3
Example Secnidazole Vaginal Soft Gelatin Capsule (100
mg/300 mg/400 mg/500 mg secnidazole dose) Fill Compositions
with Constant Vaginal Soft Gelatin Capsule Size of Size
30 Oval With A Target Fill Weight Of 1.33 g

| | Secnidazole Dose (mg) | | | |
|---|---|---|---|---|
| | 100 | 300 | 400 | 500 |
| | Vaginal Soft Gelatin Capsule Fill Composition | | | |
| Component | w/w % | w/w % | w/w % | w/w % |
| Secnidazole | 7.54 | 22.62 | 30.16 | 37.70 |
| (as a hemi-hydrate) | | | | |
| Oleoyl polyoxyl-6 | 91.53 | 76.45 | 68.91 | 61.37 |
| glycerides* | | | | |
| Polysorbate 80 | 0.43 | 0.43 | 0.43 | 0.43 |
| Lecithin | 0.50 | 0.50 | 0.50 | 0.50 |
| Vaginal Soft | 30 | 30 | 30 | 30 |
| Gelatin Capsule | | | | |
| Size (Oval) | | | | |

*Labrafil M1944 CS

TABLE 4
Example Secnidazole Vaginal Soft Gelatin Capsule (1
mg/35 mg/50 mg/75 mg secnidazole dose) Fill Compositions
with Constant Vaginal Soft Gelatin Capsule Size of
Size 30 Oval With A Target Fill Weight Of 1.33 g

| | Secnidazole Dose (mg) | | | |
|---|---|---|---|---|
| | 1 | 35 | 50 | 75 |
| | Vaginal Soft Gelatin Capsule Fill Composition | | | |
| Component | w/w % | w/w % | w/w % | w/w % |
| Secnidazole | 0.075 | 2.64 | 3.77 | 5.65 |
| (as a hemi-hydrate) | | | | |
| Oleoyl polyoxyl-6 | 98.99 | 96.43 | 95.30 | 93.42 |
| glycerides* | | | | |
| Polysorbate 80 | 0.43 | 0.43 | 0.43 | 0.43 |
| Lecithin | 0.50 | 0.50 | 0.50 | 0.50 |
| Vaginal Soft | 30 | 30 | 30 | 30 |
| Gelatin Capsule | | | | |
| Size (Oval) | | | | |

*Labrafil M1944 CS

Various surfactants (such as a nonionic surfactant) and suspending aides (such as glycerophospholipids and/or a nonionic surfactant) can be used in fill material compositions for soft gelatin capsule manufacturing. Also, different vaginal soft gelatin capsule sizes (e.g., size 30 oval capsule or a size 40 oval capsule) can be used herein.

Depending on the processability of soft gelatin capsule fill compositions with low secnidazole content, additional viscosity-modifying excipients such as polyethylene glycol 3350 can be included in the composition to increase the fill viscosity.

The soft gelatin capsule compositions having doses of from 1 mg to about 500 mg have lower adverse effects without affecting its efficacy, compared to the capsule composition having doses in a range of from about 500 mg to 1,000 mg. Local concentrations of vaginally administered secnidazole are higher than that achievable with oral administration of the same dosages without the equivalent systemic exposures as would be seen from oral administration. In addition, lower dosages can allow for smaller ovules to be developed, with increased patient acceptance.

The soft gelatin capsule compositions having doses of from 1,000 mg to about 1,500 mg are more efficacious in recurrent bacterial vaginosis or with more resistant infections, compared to the capsule composition having doses in a range of from about 500 mg to 1,000 mg.

Unless otherwise indicated, all numbers expressing quantities of ingredients, properties such as molecular weight, reaction conditions, and so forth used in the specification and claims are to be understood as being modified in all instances by the term “about.” Accordingly, unless indicated to the contrary, the numerical parameters set forth in the specification and attached claims are approximations that can vary depending upon the desired properties sought to be obtained by the present invention. At the very least, and not as an attempt to limit the application of the doctrine of equivalents to the scope of the claims, each numerical parameter should at least be construed in light of the number of reported significant digits and by applying ordinary rounding techniques. Notwithstanding that the numerical ranges and parameters setting forth the broad scope of the present invention are approximations, the numerical values set forth in the specific examples are reported as precisely as possible. Any numerical value, however, inherently contains certain errors necessarily resulting from the standard deviation found in their respective testing measurements.

Recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein is intended merely to better illuminate the present invention and does not pose a limitation on the scope of the present invention otherwise claimed. No language in the specification should be construed as indicating any non-claimed element essential to the practice of the present invention.

Groupings of alternative elements or embodiments of the present invention disclosed herein are not to be construed as limitations. Each group member can be referred to and claimed individually or in any combination with other members of the group or other elements found herein. It is anticipated that one or more members of a group can be included in, or deleted from, a group for reasons of convenience and/or patentability. When any such inclusion or deletion occurs, the specification is deemed to contain the group as modified thus fulfilling the written description of all Markush groups used in the appended claims.

Certain embodiments of this invention are described herein, including the best mode known to the inventors for carrying out the present invention. Of course, variations on these described embodiments will become apparent to those of ordinary skill in the art upon reading the foregoing description. The inventor expects skilled artisans to employ such variations as appropriate, and the inventors intend for the present invention to be practiced otherwise than specifically described herein. Accordingly, this invention includes all modifications and equivalents of the subject matter recited in the claims appended hereto as permitted by applicable law. Moreover, any combination of the above-described elements in all possible variations thereof is encompassed by the present invention unless otherwise indicated herein or otherwise clearly contradicted by context.

In all of the foregoing embodiments disclosed herein, it is to be understood that all embodiments can be further limited by using “consisting of” or “consisting essentially of” language, rather than “comprising”. When used, the transition term “consisting of” excludes any element, step, or ingredient not specified in the claims. The transition term “consisting essentially of” limits the scope of a claim to the specified materials or steps and those that do not materially affect the basic and novel characteristic(s). Embodiments of the present invention so claimed are inherently or expressly described and enabled herein.

In closing, it is to be understood that the embodiments of the present invention disclosed herein are illustrative of the principles of the present invention. Other modifications that may be employed are within the scope of the present invention. Thus, by way of example, but not of limitation, alternative configurations of the present invention may be utilized in accordance with the teachings herein. Accordingly, the present invention is not limited to that precisely as shown and described.

Although the subject matter has been described in terms of exemplary embodiments, it is not limited thereto. Rather, the appended claims should be construed broadly, to include other variants and embodiments, which may be made by those skilled in the art.

What is claimed is:

1. A soft gelatin capsule composition for vaginal administration of secnidazole comprising a therapeutically effective amount of secnidazole dispersed in a pharmaceutically acceptable carrier, wherein the therapeutically effective amount of secnidazole is a range of from about 1 mg to about 500 mg or from about 1,000 mg to about 1,500 mg.

2. The soft gelatin capsule composition of claim 1, wherein the pharmaceutically acceptable carrier is an excipient comprising a fatty acid derivative.

3. The soft gelatin capsule composition of claim 2, wherein the fatty acid derivative is a derivative of a mono-unsaturated fatty acid.

4. The soft gelatin capsule composition of claim 2, wherein the excipient comprises a polyoxylglyceride of a mono-unsaturated fatty acid.

5. The soft gelatin capsule composition of claim 4, wherein the polyoxylglyceride is selected from the group consisting of mono-unsaturated glycerides, monoglycerides oleic acid (C18:1), diglycerides oleic acid (C18:1), triglycerides oleic acid (C18:1), mono-polyethylene glycol-6 esters of oleic acid (C18:1), di-polyethylene glycol-6 esters of oleic acid (C18:1), and any combination thereof.

6. The soft gelatin capsule composition of claim 2, wherein the fatty acid derivative is a derivative of a saturated fatty acid.

7. The soft gelatin capsule composition of claim 6, wherein the excipient comprises medium chain triglycerides selected from the group consisting of caprylic (C8) acid, capric (decanoic, C10) acid, and any combination thereof.

8. The soft gelatin capsule composition of claim 2, wherein the fatty acid derivative is selected from the group comprising oleoyl polyoxyl-6 glycerides, triglycerides of caprylic and capric acids; or a combination thereof.

9. The soft gelatin capsule composition of claim 1, wherein the therapeutically effective amount of secnidazole is about 35 mg.

10. The soft gelatin capsule composition of claim 1, wherein the therapeutically effective amount of secnidazole is about 50 mg.

11. The soft gelatin capsule composition of claim 1, wherein the therapeutically effective amount of secnidazole is about 75 mg.

12. The soft gelatin capsule composition of claim 1, wherein the therapeutically effective amount of secnidazole is about 100 mg.

13. The soft gelatin capsule composition of claim 1, wherein the therapeutically effective amount of secnidazole is about 150 mg.

14. The soft gelatin capsule composition of claim 1, wherein the therapeutically effective amount of secnidazole is about 200 mg.

15. The soft gelatin capsule composition of claim 1, wherein the therapeutically effective amount of secnidazole is about 300 mg.

16. The soft gelatin capsule composition of claim 1, wherein the therapeutically effective amount of secnidazole is about 400 mg.

17. The soft gelatin capsule composition of claim 1, wherein the therapeutically effective amount of secnidazole is about 450 mg.

18. The soft gelatin capsule composition of claim 1, wherein the therapeutically effective amount of secnidazole is about 1,100 mg.

19. The soft gelatin capsule composition of claim 1, wherein the therapeutically effective amount of secnidazole is about 1,200 mg.

20. The soft gelatin capsule composition of claim 1, wherein the therapeutically effective amount of secnidazole is about 1,300 mg.

21. The soft gelatin capsule composition of claim 1, wherein the therapeutically effective amount of secnidazole is about 1,400 mg.

22. The soft gelatin capsule composition of claim 1, wherein the therapeutically effective amount of secnidazole is about 1,500 mg.

23. The soft gelatin capsule composition of claim 1, wherein the therapeutically effective amount of secnidazole is in a range of from about 1 mg to about 400 mg or from about 1,100 mg to about 1,500 mg.

24. A method for treating bacterial vaginosis in a subject in need thereof comprising:
selecting the subject in need of treatment for bacterial vaginosis; and
administering to the subject a soft gelatin capsule composition by vaginal route comprising a therapeutically effective amount of secnidazole,
wherein the therapeutically effective amount of secnidazole is in a range of from about 1 mg to about 500 mg or from about 1,000 mg to about 1,500 mg

25. The method of claim 24, wherein the subject is a human, a human female or a pregnant human female.

26. The method of claim 24, wherein the therapeutically effective amount of secnidazole is in a range of from about 1 mg to about 400 mg or from about 1,100 mg to about 1,500 mg.

27. A method for treating trichomoniasis in a subject in need thereof comprising:
selecting the subject in need of treatment for trichomoniasis; and
administering to the subject a soft gelatin capsule composition by vaginal route comprising a therapeutically effective amount of secnidazole,
wherein the therapeutically effective amount of secnidazole is in a range of from about 1 mg to about 500 mg or from about 1,000 mg to about 1,500 mg.

28. The method of claim 27, wherein the subject is a human, a human female or a pregnant human female.

29. The method of claim 27, wherein the therapeutically effective amount of secnidazole is in a range of from about 1 mg to about 400 mg or from about 1,100 mg to about 1,500 mg.
